(19) United States
(12) Patent Application Publication
(10) Pub. No.: US 2011/0282190 A1
(43) Pub. Date: Nov. 17, 2011

(54) COMBINED ENDOSCOPIC SURGICAL TOOLS

(75) Inventors: Sean Caffey, Manhattan Beach, CA (US); Jeffrey Brennan, Los Angeles, CA (US); Mark Humayun, Glendale, CA (US)

(73) Assignee: oProbe, LLC, Pasadena, CA (US)

(21) Appl. No.: 13/106,390

(22) Filed: May 12, 2011

Related U.S. Application Data
(60) Provisional application No. 61/334,821, filed on May 14, 2010.

(51) Int. Cl. A61B 6/00

(52) U.S. Cl. 600/427

(57) ABSTRACT

A probe assembly includes a forward-imaging optical coherence tomography (OCT) probe having a field of view and an instrument, adjacent to the probe, for performing manipulations at least within the OCT field of view.

### CROSS-REFERENCE TO RELATED APPLICATIONS

This application claims priority to and the benefit of, and incorporates herein by reference in their entireties, U.S. Provisional Patent Application No. 61/334,821, which was filed on May 14, 2010, the entire disclosure of which is hereby incorporated by reference.

### TECHNICAL FIELD

In various embodiments, the present invention relates generally to surgical tools, in particular to tools utilizing an optical coherence tomography (OCT) probe.

### BACKGROUND

Surgical endoscopes can be adapted to hold and actuate a variety of different surgical instruments while imaging the region of interest using video, ultrasound, laser, optics or other imaging modalities. The goal of many surgeries is to place small, delicate implants such as shunts, stents, drug-coated rods, or other medical devices within a specific orientation of a body cavity in order to minimize complications and maximize the implant's desired effect. Surgeons currently use a variety of instruments such as grippers, scissors, or introducers to manipulate the implant inside the body. In order to gain access to specific tissues or to perform delicate dissection without cutting nerves or arteries, proper visualization or planned visualization by pre-op imagery by the surgeon is required to minimize complications. To understand and visualize the area of interest for the implant before or during the operation, surgeons currently use computed tomography (CT), magnetic resonance imaging (MRI), ultrasound, and other imaging modalities. Biliary stenting surgery, for example, is used to treat obstructions that occur in the bile ducts, and in this procedure physicians use real-time imaging with tools to improve the placement of the stents. A biliary stent is a thin, tube-like structure that is used to support a narrowed part of the bile duct and prevent the reformation of the stricture. Biliary stenting surgery involves a multitude of imaging equipment, pre-operative planning, and intra-operative endoscopy in order to guide the small-diameter stent to the proper location within the biliary tube. A combination of surgical endoscopes, which can provide better visualization and real-time feedback, and a surgical instrument is thus necessary to assist the surgeon in placing the implant.

Orthopedic surgeries also require the ability to visualize structures (e.g., cartilage) in joints such as the knee. Tools such as cutters, grippers, or radio-frequency (RF) ablation devices may be used to cut or manipulate the tissue during procedures. Prostatectomies involve the surgical removal of all or part of the prostate gland. A cystoscope is passed up the urethra to the prostate, where the surrounding prostate tissue is excised. Hook electrocautery, bipolar forceps, and scissors are used to dissect the medial umbilical ligament, for example, and the lateral peritoneum to gain access to the prostate. The ability to visualize the veins and nerves would help surgeons avoid them during cautery.

Glaucoma surgeries are minimally invasive procedures used to lower intraocular pressure by either removing part of the eye's anatomy (such as the trabecular meshwork and adjacent structures) or implanting shunts to bypass the traditional outflow of aqueous humor through the Schlemm's Canal. The ability to visualize the local anatomy before and after the placement of the shunt would assist significantly in such procedures.

In these and other surgical procedures, MRI, CT, ultrasound, and confocal microscopy are commonly used to visualize tissues before and during surgery. OCT is another popular imaging modality commonly employed in ophthalmology to non-invasively image the anterior or posterior structures of the eye. OCT provides high-resolution, real-time, cross-sectional, and subsurface tomographic imaging of the microstructure in materials and biologic systems by measuring back-reflected infrared light. OCT provides advantages over ultrasound and MRI in that OCT can provide morphological tissue imaging; OCT also has advantages over confocal microscopy in that confocal microscopy cannot provide millimeter-deep morphology. OCT has been used for biomedical applications where many factors affect the feasibility and effectiveness of any imaging technique. OCT as traditionally performed is a non-invasive, non-contact, transpupillary imaging technology which can image, for example, retinal structures in vivo with a high resolution. Recently, Fourier-domain, quantum and full-field OCT have gained popularity due to an increase in signal-to-noise ratio and decrease in imaging time.

OCT has transformed the field of ophthalmology and promises to have a similar impact on a variety of other medical specialties. A particular mode of OCT, termed “A-scan,” provides one-dimensional axial depth scans of the tissue of interest, thus providing information on the identity, size, and depth of subsurface features. A series of spatially adjacent A-scans (typically lying in a straight line) may be combined to form a two-dimensional reconstructed image of the imaged area (termed a “B-scan”), offering surgeons a visual reconstruction of subsurface features. Likewise, three-dimensional images, termed “C-scans,” may be formed by “stacking” multiple B-scans.

Cross-sectional images of the posterior or anterior structures of the eye are produced by OCT scanning using the optical back-reflecting of light in a fashion analogous to B scan ultrasonography. The anatomic layers within the retina can be differentiated, retinal thickness can be measured, and the appearance of a variety of posterior segment pathologies, including diabetic retinopathy, macular holes, epiretinal membranes, cystoid macular edema, central serous choroidopathy, and optic disc pits can be studied. A problem with traditional extraocular OCT scanners is that many of the long wavelengths (e.g., 1310 nm) are significantly attenuated before reaching the retina due to the light absorption by the cornea and the fluid in the anterior chamber. Long wavelengths thus are ineffective for extraocular retinal imaging even though they provide better tissue penetration.

At present, scanning with modalities such as OCT is not easily integrated with the surgical procedure itself. Inserting an OCT probe along with surgical instruments can crowd the target space, and each additional tool placed in the patient's body increases both risk and the invasiveness of the procedure.

### SUMMARY

The present invention combines OCT imaging and surgical manipulation capabilities into a single tool, allowing surgeons to improve their implantation times and the safety of the overall implantation and dissection. The combination of a probe assembly, including a forward-imaging OCT needle probe and surgical instruments such as forceps, scissors, or cautery equipment can provide substantial assistance for the surgeon in performing surgeries. The probe provides the surgeon with an intraocular, forward-looking, angled-looking, or side-looking OCT image of the tissue, permitting assessment of the area (e.g., for the presence of arteries, veins, nerves, or devices for aiding contrast) and performing manipulations in the same field of view of the OCT image in a more accurate and controlled manner. In addition, the combination tool achieves deeper tissue penetration for imaging since long wavelength light from the OCT probe is emitted directly on the intraocular target without being absorbed by the cornea or the fluid in the anterior chamber as it propagates.

In various embodiments, the OCT element utilizes the paired-angle rotation scanning OCT (PARS-OCT) configuration and is capable of A-scans, B-scans, or C-scans. A single fiber utilizing an A-scan combined with surgical instruments can provide an inexpensive and smaller device for confirmatory A-scan data during surgery. Devices in accordance with the invention may be utilized in combination surgeries so the probe is accommodated within existing surgical incisions or holes. Embodiments of the invention may be deployed in robotic surgery environments.

Accordingly, in one aspect, the invention pertains to a probe assembly comprising an elongated member insertable through an incision in a patient's body. In various embodiments, the probe assembly comprises an OCT probe having a field of view, and an instrument, adjacent to the probe, for performing manipulations at least within the OCT field of view. In various embodiments, the OCT probe provides forward imaging, side imaging, and/or angled imaging.

In one embodiment, the instrument is configured for movement from a body of the probe assembly into the field of view of the OCT probe; in some embodiments, the instrument is configured for arcuate extension from the probe assembly into the field of view. The instrument and the OCT probe may, for example, be contained within the body. The manipulations may comprise cutting, gripping, cauterizing and/or ablating. In some embodiments the instrument is a set of retractable grippers.

In some embodiments, the instrument is configured to manipulate tissue and/or an implant within the field of view of the OCT probe. In various implementations, the instrument comprises or consists of a set of forceps. In other embodiments, the instrument comprises or consists of an extendible and directional moving element for facilitating rotation and orientation of the implant. The instrument may, for example, be configured to deploy an implant within the field of view of the OCT probe.

In various embodiments, the instrument comprises or consists of the output of a laser, an applicator for an implantable item, a cutter, an RF ablation probe, a cautery tool, and/or a retractable blade. The retractable blade may be made of metal, silicon, ceramic, and/or plastic.

In a second aspect, the invention pertains to a system comprising a probe assembly that comprises an elongated member insertable through an incision in a patient's body, an OCT imaging engine coupled to the OCT probe, and a user-controlled actuator. In various embodiments, the probe assembly comprises an OCT probe that has a field of view, and an instrument, adjacent to the probe, for performing manipulations at least within the OCT field of view. In various embodiments, the OCT probe may provide forward imaging, side imaging, and/or angled imaging.

In some embodiments, an actuator for controlling the operation of the instrument is located remotely from the instrument and is operatively coupled to the instrument. In one implementation, the actuator communicates pneumatically with the instrument along a fluid path. In another implementation, the actuator communicates mechanically with the instrument. For example, the actuator may be located on a user handle, and a wire may connect the actuator to the instrument to facilitate extension and retraction thereof.

In a third aspect, the invention relates to a method for performing manipulations during surgery. In various embodiments, the method comprises inserting a probe assembly adjacent to or into a target, capturing an OCT image of the target via the probe, which has a field of view, and performing manipulations on tissue and/or an implantable device within the field of view of the OCT probe. In various implementations, the OCT image is obtained via image-gathering optics and the manipulations are performed by an instrument; the image-gathering optics and the instrument may, for example, be contained within the probe assembly. The manipulations may comprise cutting, gripping, cauterizing, and/or ablating the target.

These and other objects, along with advantages and features of the present invention herein disclosed, will become more apparent through reference to the following description, the accompanying drawings, and the claims. Furthermore, it is to be understood that the features of the various embodiments described herein are not mutually exclusive and can exist in various combinations and permutations.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which:

FIG. 1A schematically depicts a tool combining an OCT probe with an instrument.

FIG. 1B schematically depicts an instrument performing manipulations in the field of view of the OCT probe.

FIG. 1C illustrates a glaucoma shunt that is manipulated in the anterior chamber of the eye by a tool in accordance herewith.

FIG. 2A depicts a flexible and extendible moving portion of an instrument that communicates with a remote actuator; the moving portion is controlled by a mechanical element.

FIG. 2B depicts a tool combining an OCT needle probe with two shape-memory alloy wires.

FIG. 3A is a sectional view of a foley catheter filled with fluid for providing good contrast for the OCT probe; the catheter communicates pneumatically with an actuator along the fluid path.

FIG. 3B depicts an OCT probe placed adjacent to or behind an intraocular lens for providing real-time feedback during the implantation.

FIGS. 4A and 4B are elevational views of a tool combining an OCT probe with a blade and a cautery device, respectively.

FIG. 5 depicts a tool combining an OCT probe with a pair of cutters, grippers, and a radio-frequency ablation device.

FIG. 6A illustrates a combination tool entering the duodenum through the same pathway as a percutaneous endoscopic gastrostomy (PEG) tube to allow the surgeon to visualize the biliary stricture.

FIG. 6B illustrates two methods of using a combination tool during percutaneous transhepatic cholangiography (PTC): (i) the combination tool is inserted into a needle introduced into the bile duct, and (ii) the combination tool is inserted into the bile duct via a small incision used for laparoscopy.

FIG. 7A depicts a combination tool and a catheter filled with fluid for providing good contrast during prostatectomy.

FIG. 7B depicts a flexible shaft incorporating a combination tool integrated into the catheter for aiding the insertion of the integrated tool into the prostate.

FIG. 7C illustrates a laser and an OCT probe for laser ablation surgery.

FIG. 8A depicts a trabecular bypass shunt implanted inside the eye to direct the outflow of aqueous humor.

FIG. 9A depicts a translimbal implant used for glaucoma drainage.

FIG. 9B illustrates a translimbal implant placed under a scleral flap in the eye.

FIG. 10 illustrates a gold microshunt implanted between the choroid and sclera.

FIG. 11 depicts an Aquashunt combined with the OCT probe for providing real-time feedback during surgery.

### DETAILED DESCRIPTION

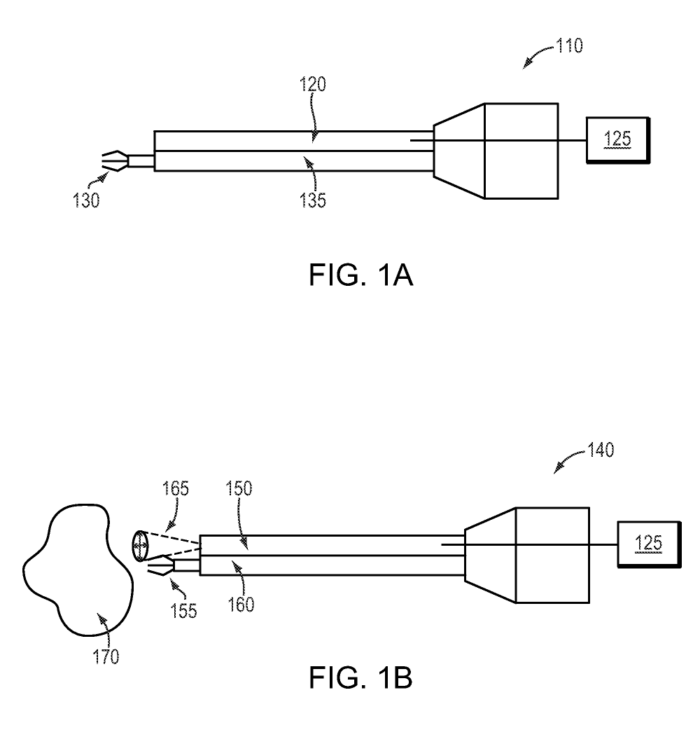

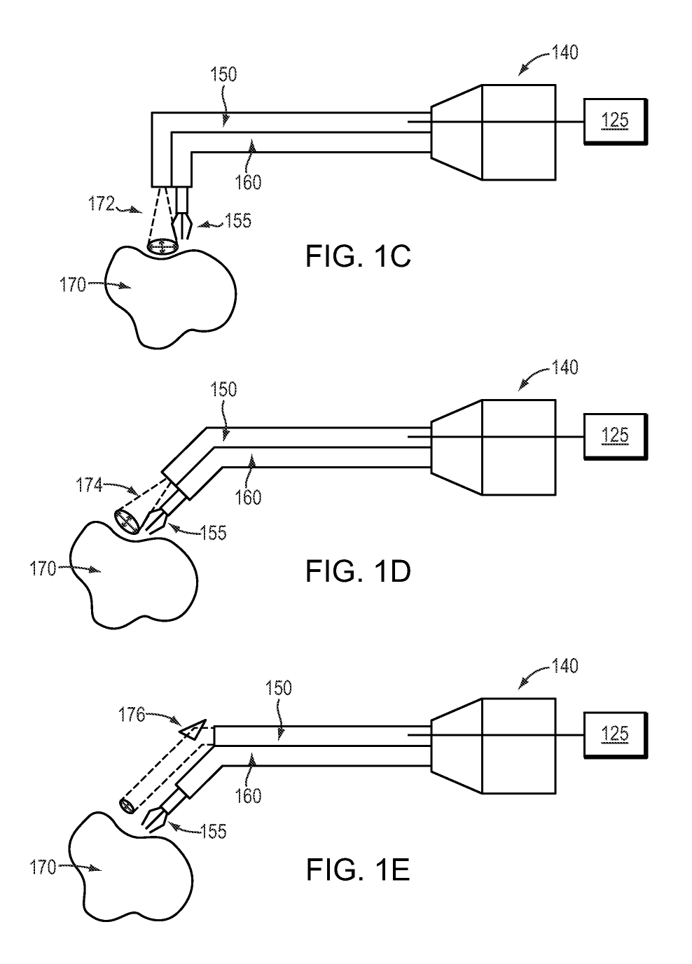

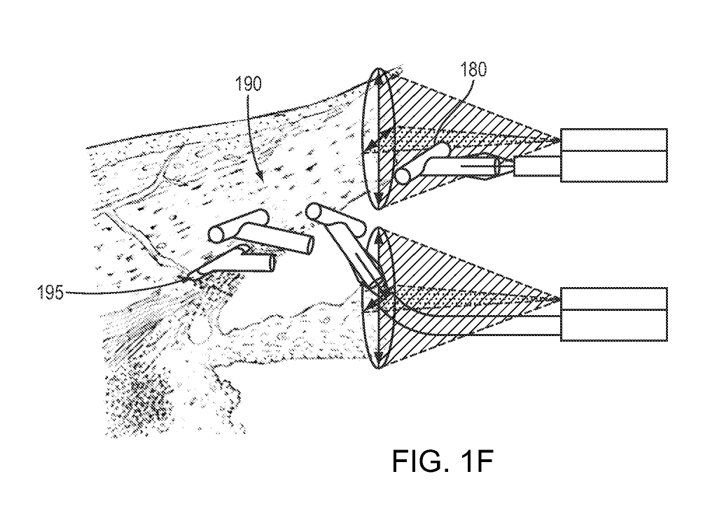

In various embodiments, as illustrated in FIG. 1A, combination tools 110 in accordance herewith are handheld instruments that combine either an A scan or B scan OCT probe contained in a lumen 120, which in use is optically coupled to a conventional OCT imaging engine 125, together with an instrument 130 (such as picks, scissors, cautery, or ablation tools) for gripping a device or manipulating tissue during surgery that is retractable within a lumen 135. In some embodiments, a single lumen is used, while in other embodiments, the lumens 120, 135 are partitions within a single sleeve. In still other embodiments, lumens 120, 135 are separate sleeves. It should be stressed that lumens 120, 135 may be longer than the figures suggest; they are illustrated in the manner depicted for simplicity.

With reference to FIG. 1B, the device 140 includes a forward-imaging OCT needle probe 150 utilizing PARS-OCT and capable of B scans, and a set of forceps 155 retractable and extendible within the lumen 160 of the instrument. The device 140 assists the surgeon in placing implants such as stents, drug-delivery elutional implants (e.g., a dexamethesone drug eluting pellet for a diabetic macular edema or a triamcinolone eluting device attached to the pars plana inside the eye), or other implants. Embodiments of the invention assist the surgeon with a forward-looking OCT image 165 of the tissue 170, enabling assessment of the local anatomy. Referring to FIGS. 1C, 1D, and 1E, in some embodiments the OCT needle probe provides side-imaging 172 and/or angled imaging 174 and 176 (e.g., 45 degrees) relative to the target; this is useful when the OCT probe is inserted at a tangential angle and not directly in front of the target. Side imaging and angled imaging can be provided by directing light emitted from the OCT probe using a suitably shaped lumen 150 (FIGS. 1C and 1D) or using an optical element 176 (e.g., a prism or a mirror) as shown in FIG. 1E. FIG. 1C depicts a clinical use of the combination tool, where a glaucoma shunt 180 is manipulated in the anterior chamber of the eye 190 while the collecting channel 195 within the Canal of Schlemm is visualized at the same time.

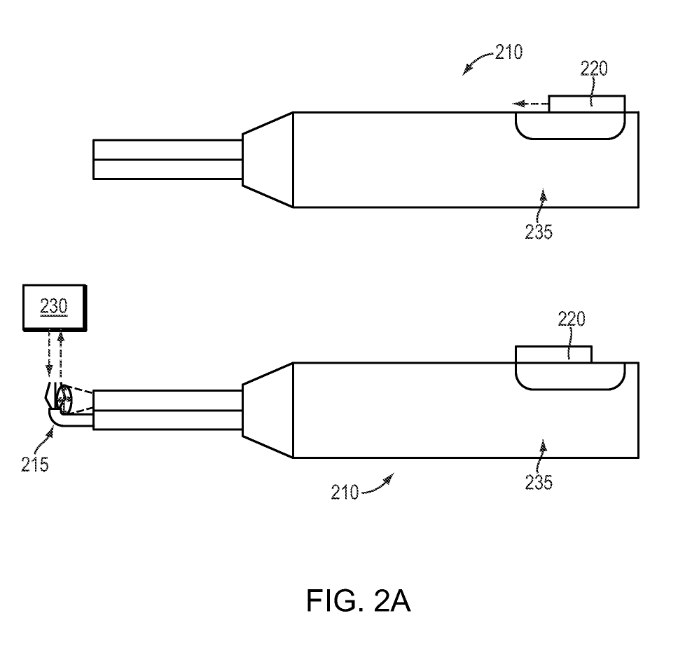

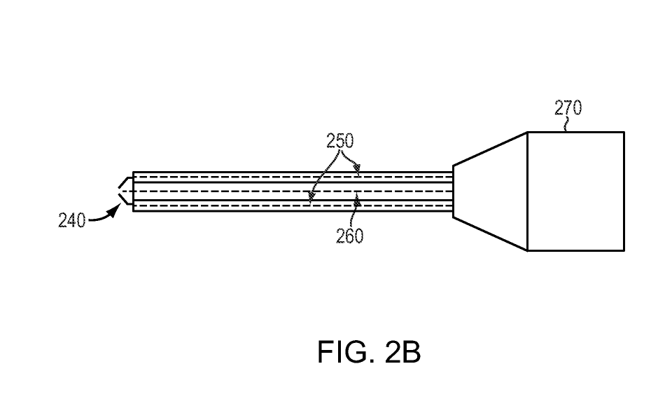

FIG. 2A depicts additional embodiments of the device 210 including a flexible and extendible moving portion 215, controlled by a mechanical element 220, and giving the surgeon additional degrees of movement to rotate and orient the implant being positioned. Shape-memory alloys such as Nitinol are biocompatible and may be used in a flexible wire, stent, or smart material as the moving portion 215. A shape-memory “remembers” its original, cold-forged shape and returns to the pre-deformed shape upon heating. One of the advantages of using a shape-memory alloy is the high level of recoverable plastic strain that can be included. The maximum recoverable strain the alloy can hold without permanent damage may be, for example, 8%, much larger than conventional steels with a maximum strain of, e.g., 0.5%. Therefore, the shape-memory alloy can be manufactured in almost any shape and size. Shape-memory alloys can thus be used to provide directional movement to the surgeon by extending the instrument (e.g., forceps) in a specific, controlled movement. A remote actuator 230 can be used further to open and close e.g., the forceps. In one embodiment, the actuator communicates mechanically with the instrument 215. In another embodiment, the actuator is located on the handle 235, facilitating extension and retraction of the instrument 215 via thumb operation, e.g., by means of a wire connecting the actuator to the instrument. With reference to FIG. 2B, in another embodiment, two retractable shape-memory alloy wires 240 protected by and movable within outer sleeves 250 are combined with a PARS-OCT imaging needle 260 to work as OCT-enabled pincers in the device 270. For example, the sleeves 250 and an optical fiber leading to the imaging needle 260 may be contained within a surrounding outer sleeve, or may be separate but attached elements.

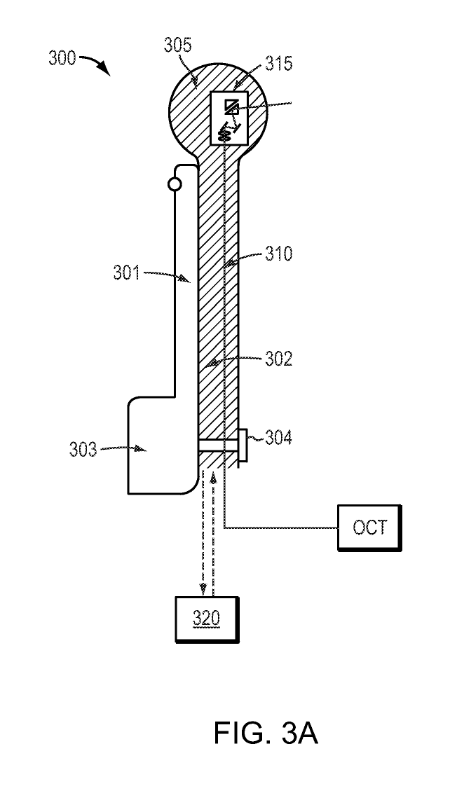

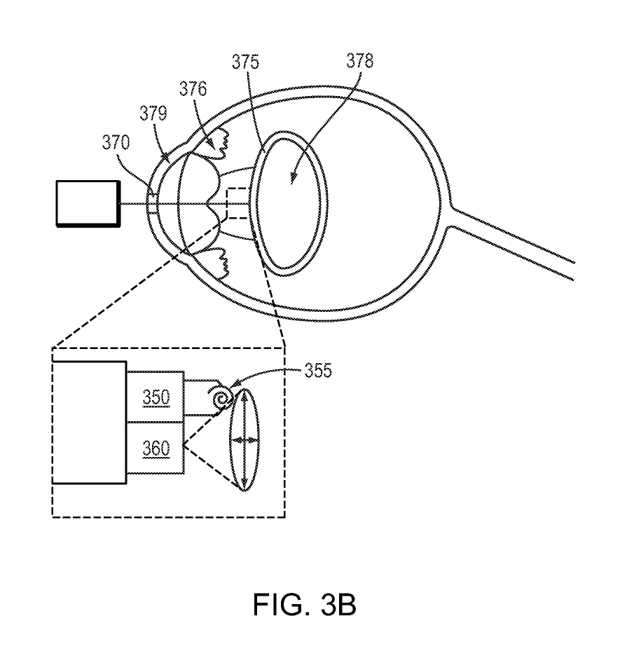

It is possible to utilize a closed-end catheter inflated with gas or liquid near the area of interest for dissection or implantation in order to improve the contrast of the anatomical region. For example, placing a foley catheter into a male patient's urethra and filling it with water or gas provides better contrast, and allows an OCT probe to easily image the location of the urethra. Referring to FIG. 3A, a foley catheter 300 has two separated channels 301, 302 running down its length. One channel 301 is open at both ends, allowing urine to drain out into a collection bag 303, while the other channel 302 has a valve 304 on the outside end and connects to a balloon 305 at the tip. During operation, channel 302 and balloon 305 may be inflated with water or gas, providing good contrast for the OCT probe 310 (e.g., an optical fiber) to image the urethra and bladder. The OCT probe 310 may include optical elements 315 (e.g., mirrors, prisms, or side-scanning lenses) to redirect light and enable side or angled viewing of the urethra and bladder in addition to forward viewing. Without the use of such a catheter, certain cavities in the body (such as the urethra) are only a “potential” space or pseudospace which, when unoccupied, cannot be distinguished from the surrounding tissues. Mechanical actuation of the OCT-enabled tools, such as the OCT scanning mechanism for a B-scan or C-scan or movement of the instrument (e.g., the catheter), can be achieved pneumatically along the fluid path 302 from the same pneumatic systems 320 that power other surgical tools. In this manner, a surgeon can voice-activate or use a foot pedal, for example, to create different actuations such as forceps grip or release. In other embodiments, these functions can be gear-driven or operated by other mechanical couplings.

FIG. 3B illustrates an insertion tool for implanting the intraocular lens (IOL) in the lens capsule that is combined, in accordance herewith, with an OCT probe providing A-scan, B-scan, and/or C-scan capabilities. The IOL is an implant for replacing the existing clouded crystalline lens due to a cataract or as a form of refractive surgery to change the eye's optical power. During surgery, the insertion tool 350 carrying the IOL 355 (usually rolled up for easy introduction into the anterior chamber) and the combined OCT probe 360 (located adjacent to or behind the IOL) is inserted through a small incision 370 into the lens capsule 375. The OCT probe ensures that the IOL is deployed and placed correctly and that there are no tears in the capsule. The OCT probe also allows the surgeon to optimally evaluate structures such as the iris 376, lens 378, and the cornea 379 after the IOL 355 is implanted. Furthermore, the OCT probe 360 can be used to measure biometrics of the eye, including the iridocorneal angle or the surface features of the cornea, for example, to improve the outcome of photoablation, corneal sculpting, and other procedures.

Microdevices using microelectromechanical systems (MEMS) may be added to the combination tools in accordance herewith. MEMS processes to build small surgical tools include the “EFAB” process described below. Based on multi-layer electroplating and planarization of metals, this process can be used to provide extremely sharp instruments. The very small dimensions it permits facilitate production of tools that allow surgeons to mechanically cut, cauterize, pinch or dissect tissues during surgery with great accuracy.

The EFAB process forms multiple independently patterned metal layers to create complex three-dimensional (3D) constructs with micron-level precision, faciliating design and creation of new RF, optical and inertial micro-devices more quickly and with a broad range of functionality. EFAB technology is a flexible and versatile process that can create complex 3D microdevices composed of dozens (e.g., 50) layers—where, by comparison, conventional MEMS processes may be limited to five layers—that are otherwise impossible or impractical to make. A representative EFAB process involves three steps: patterned layer deposition, blanket layer deposition, and planarization.

In the first step, a layer of metal is deposited into a pattern corresponding to the cross-section of the fabricated device. A second material is then electroplated onto the substrate to completely cover the first layer, and this is subsequently planarized to form a two-material layer. The same process is repeated several times until all cross-sections of the 3D design have been constructed. After the layers are formed, an etchant removes the first layer of metal, leaving behind the free-standing final structure. Finally, the structure is assembled into the combined tool with the optical path of the combination tool either on the side of the device, behind the device (i.e., the tool is pushed by the OCT lens system, and then OCT imaging is turned on), or integrated in the middle of the tool.

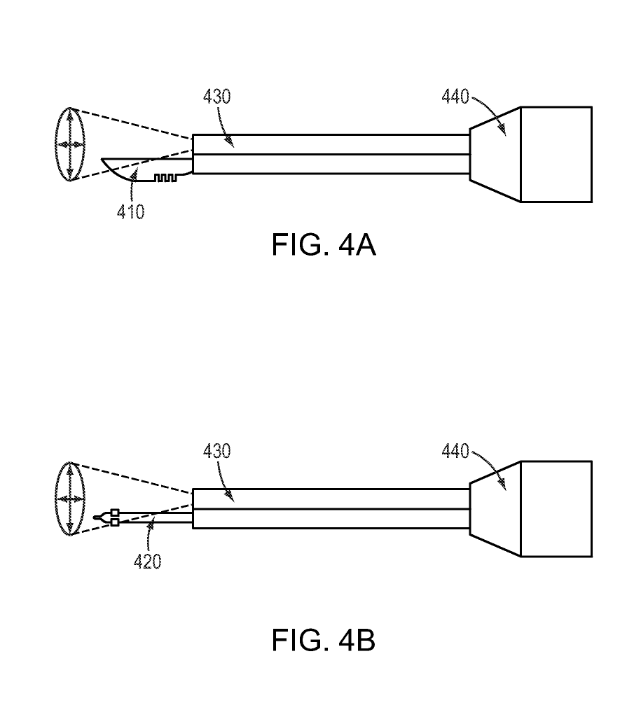

Blades made of metal, silicon, ceramic, plastic or a combination can be added to combination tools in accordance herewith. With reference to FIGS. 4A and 4B, a blade 410 or cautery device 420 each represent a simple, inexpensive element to combine with an OCT probe 430 in the combination tool 440. A surgeon will be able to easily see what he or she is cutting beyond the layers of the tissue that are currently evident only by video endoscope or pre-op imaging. Vascular surgery, for example, needs very careful attention on tissues and tissue composition in order to avoid the slicing of arteries or nerves, which both can be easily viewed by OCT. Anisotropically etched silicon blades, hardened by CVD diamond, for example, can be combined with the tool for an inexpensive precision design.

### Orthopedic Surgeries

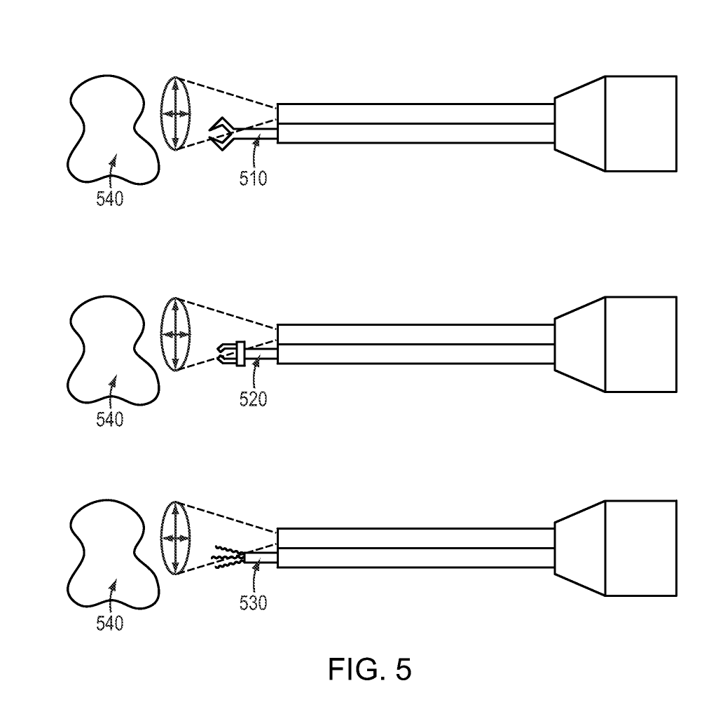

Orthopedic surgeons need a fast way to visualize structures in the knee, such as cartilage, and use an instrument (e.g., a pair of cutters 510, grippers 520 or RF ablation devices 530, as shown in FIG. 5) in order to cut or manipulate the tissue 540 during procedures such as knee arthroscopy and meniscectomy, shoulder arthroscopy and decompression, carpal tunnel releases, knee arthroscopy and chondroplasty, removal of support implants (from previous surgeries), knee arthroscopy and anterior cruciate ligament reconstruction, knee replacements, debridement of skin/muscle/bone/fractures, laminectomy (cutting through muscles to dissect into and remove the posterior spinal ligament), and low-back intervertebral disc surgeries. All of these procedures require some combination of tissue manipulation while imaging for the surgery. Using the combination tool can provide good real-time visualization while performing tissue manipulations.

### Biliary Stenting

Biliary stenting is used to treat obstructions that occur in the bile ducts and exemplifies procedures in which physicians use real-time imaging with tools to improve the placement of an implant, in this case a stent. A biliary stent is a thin, tube-like structure that is used to support a narrowed part of the bile duct and prevent the reformation of a stricture. Stents may be made of plastic or metal. Two most common methods used to place a biliary stent are endoscopic retrograde cholangiopancreatography (ERCP) and percutaneous transhepatic cholangiography (PTC).

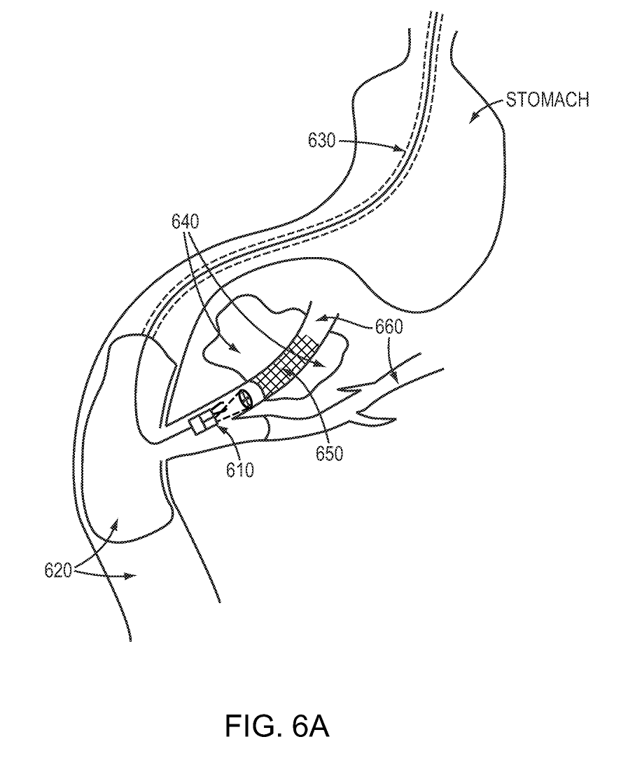

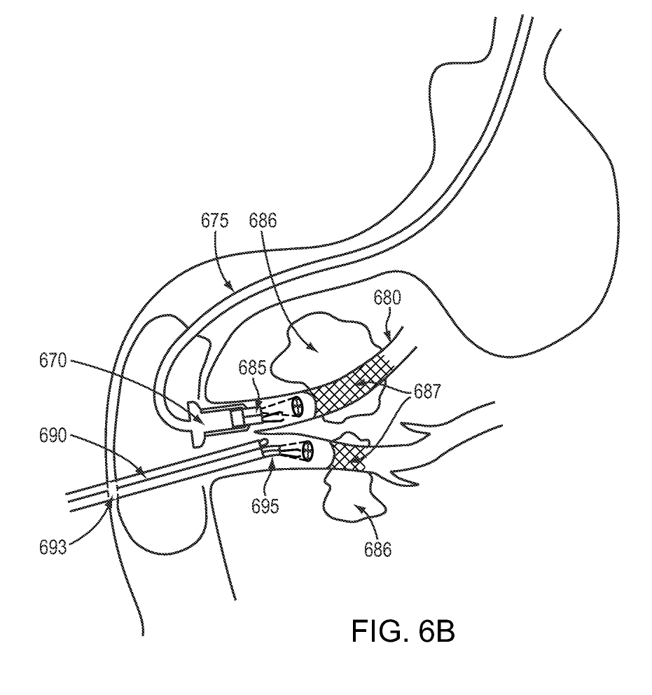

In ERCP, a series of x-rays are taken as a dye moves through the ducts. If the x-ray images show that a biliary stricture exists, a stent may be placed into a duct to relieve the obstruction. In order to achieve this, special instruments are inserted into the endoscope, and a sphincterotomy (i.e. a cut into the sphincter of Oddi) is performed to provide access to the bile ducts. In some cases, the biliary stricture may first be dilated or expanded using a catheter—i.e., a thin, flexible tube—after which a balloon-type device is inflated therein. The stent is then inserted into the bile duct. As depicted in FIG. 6A, a combination tool 610 can enter the duodenum 620 through the same pathway as a percutaneous endoscopic gastrostomy (PEG) tube 630 in order to allow the surgeon to visualize this biliary stricture 640 without the need for radiation or dyes (which can cause kidney failure in certain patients), as are common in ERCP. A biliary stent 650 is then placed into a duct 660 to relieve the obstruction.

For PTC, a thin needle is used to inject a contrast dye through the skin and into the liver or gallbladder; x-rays are then taken while the dye moves through the bile ducts. If a biliary stricture becomes evident, the combination tool can be used percutaneously during PTC in order to manipulate and place the stent. FIG. 6B illustrates two methods to accomplish this: (i) a hollow needle 670 attached to a catheter 675 is introduced into the bile duct 680, and the combination tool 685 is inserted into the needle to examine the biliary stricture 686 and place the stent 687; and (ii) a combination of laparoscopy 690, an operation performed in the abdomen through small incisions 693, with the combination tool 695 allowing the stent to be quickly placed without the need for radiation (thereby reducing any possible damage to patients with a compromised esophagus or history of gastroesophageal reflux disease (GERD)).

During these biliary or other laparoscopic surgeries, the combination tool can allow the surgeon to quickly visualize around the duct in order to avoid the cystic artery during dissection. Because the surgeon conventionally utilizes a laparoscope to visualize the procedure, the combination of an OCT probe and surgical tools can be especially advantageous in supplementing or even avoiding the need for laparoscopic imaging to perform the surgery. Other endoscopic visualization techniques, such as ultrasound, can also or alternatively be utilized with the combination tool.

In many procedures, the combination tool can be utilized to “tunnel” into delicate tissue with confidence, for example, enabling the surgeon to visually verify that an artery is not buried within a fat tissue forward of the tunneling OCT head. Therefore, a number of different shaped cautery tools can be used in order to allow the surgeon to observe tissue at the surgical site. A further advantage of the combination tool is its ability to permit visualization though bleeding areas. Long near-infrared wavelength light (e.g., 1310 nm) offers superior imaging qualities over shorter-wavelength light due to its deeper tissue penetration resulting from reduced scattering.

### Prostatectomy

A prostatectomy is the surgical removal of all or part of the prostate gland. Abnormalities of the prostate, such as a tumor, or enlargement of the gland itself for any reason, can restrict the normal flow of urine along the urethra. A cystoscope (i.e., a resectoscope which has a 30-degree viewing angle, along with a resectoscopy sheath and working element) is passed up the urethra to the prostate, where the surrounding prostate tissue is excised. The present invention improves over traditional imaging devices since the combination tool is small, made inexpensively, and has a small form factor that facilitates its introduction into small sites within the body. As opposed to catheter tools, the compact and small size of a PARS-OCT probe, for example, allows additional space for fixed devices to be built into the sleeve of the probe.

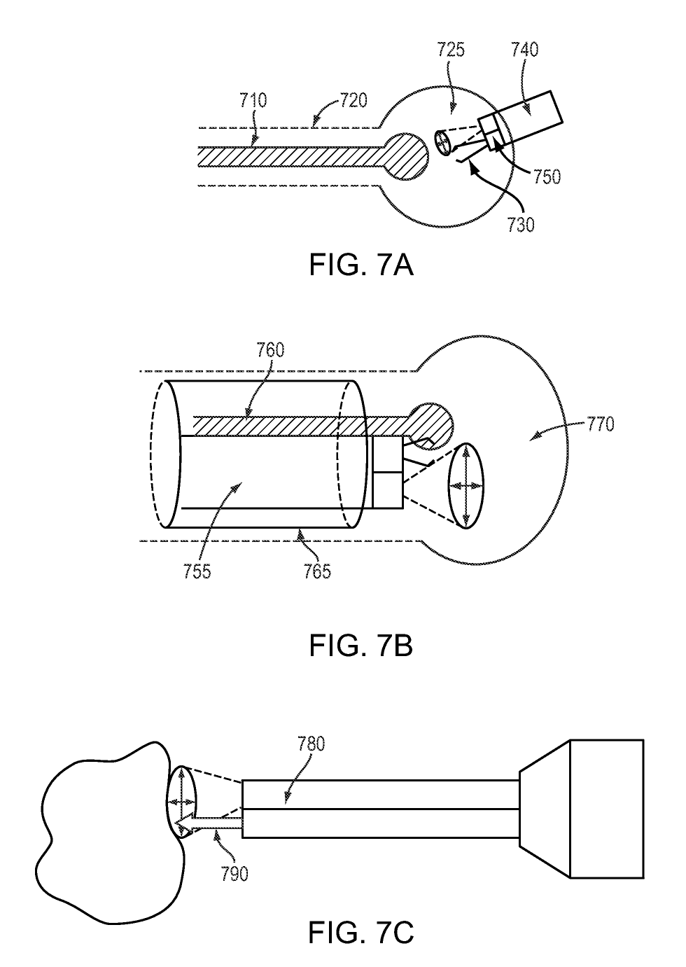

Careful dissection is used next to delicate structures such as muscle, veins, arteries, ureters, seminal vesicles, vas deferens, and nerves during prostatectomies. In order to assist the surgeon in localizing some of the anatomy, embodiments of the present invention, as depicted in FIG. 7A, may be utilized with fluid-filled catheters or balloons 710, passed up the urethra 720 to the prostate 725, in order to (i) enhance the contrast and sensitivity of the surrounding tissue (cavities filled with gas, water or dye have excellent contrast compared to surrounding body tissues) and (ii) assist the surgeon to mechanically move the tissue of interest to a desired location. A hook electrocautery, bipolar forceps, or scissors 730 are used to dissect the medical umbilical ligament, for example, and the lateral peritoneum to gain access to the prostate while protecting the superificial dorsal vein. An OCT imaging probe 740 with, for example, a cautery adapter 750 on it can help visualize the veins and nerves. In another embodiment, referring to FIG. 7B, the combination tool 755 is small enough to be integrated into the catheter 760. A flexible shaft 765 incorporating the combination tool 755 and the catheter 760 can be utilized to aid the insertion of the integrated tool into the prostate 770.

Laser prostate surgery utilizes laser energy to remove tissue. With laser prostate surgery, a fiber-optic cable pushed through the urethra is used to transmit laser light such as holmium-Nd:YAG high-powered “red” or potassium titanyl phosphate (KTP) to vaporize the adenoma. The specific advantages of utilizing laser energy rather than a traditional electrosurgical transurethral resection of the prostate (TURP) include a decrease in the relative blood loss, elimination of the risk of TUR syndrome, the ability to treat larger glands, as well as treating patients who are actively being treated with anti-coagulation therapy for unrelated diagnoses. As shown in FIG. 7C, the OCT probe 780 can be combined with a laser 790 for this added benefit.

Robotic surgery may be used to aid the surgeon with precise manipulation of tools while an endoscope helps visualize the procedure. Combination tools in accordance herewith can be used with robotic surgery to better visualize the patient's anatomy and execute surgical manipulations.

### Ophthalmology

Glaucoma is a group of diseases of the optic nerve involving loss of retinal ganglion cells in a pattern that is characteristic of a progressive optic neuropathy. Trabeculectomy remains the “gold standard” for glaucoma surgery. However, it is associated with many complications, including hypotony, choroidal effusions, overfiltration, and endophthalmitis in the immediate postoperative period, as well as failure from fibrosis, bleb-related problems (such as discomfort and leaky cystic blebs), and long-term bleb-related infections. Minimizing the trauma to the patient and improving the imaging to the surgeon allows for a less invasive surgery with the potential for better long-term outcomes.

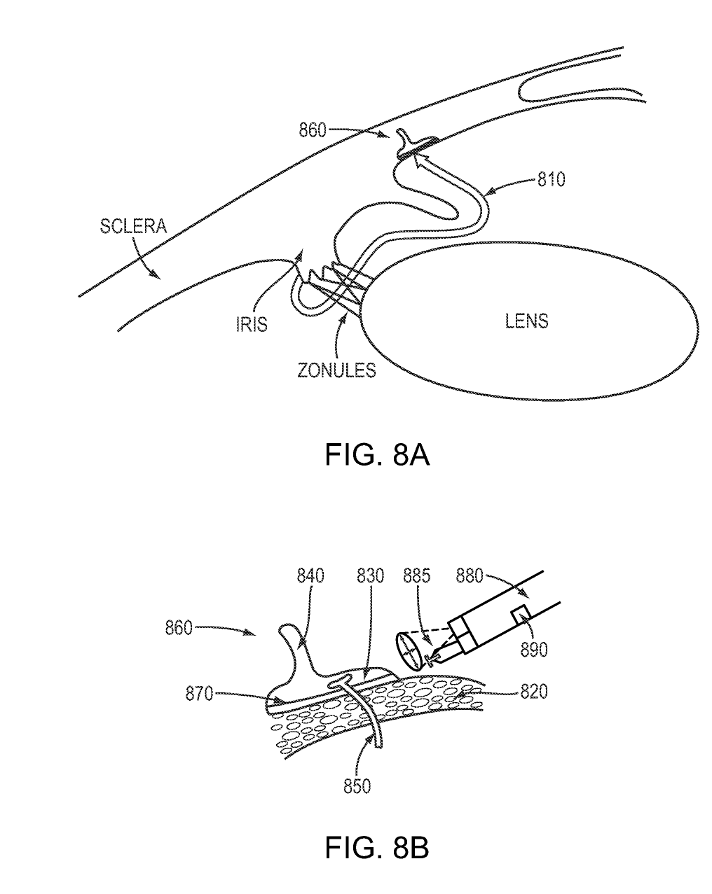

With reference to FIG. 8, the route 810 of aqueous humor drainage from the human anterior segment occurs primarily through the trabecular meshwork 820. From the trabecular meshwork 820, aqueous humor drains into Schlemm's canal 830 and empties into collector channels 840 that lead to the episcleral venous system. Newer procedures use different techniques—bypass, dilation, or ablation—to tackle obstructions but these share two features: first, they are designed to eliminate the anatomic obstruction; and second, they are intended to avoid a healing response. In other words, the area must not close again due to scarring.

Implant devices used in glaucoma surgery include direct fluid via subconjunctival space (Ahmed valve, Molteno, Baerveldt, Krupin); translimbal devices (Express Shunt), diverting the aqueous into the limbal subconjunctival space similar to trabeculectomy; direct fluid via suprachoroidal space, diverting the aqueous humor into the suprachoroidal space; direct fluid via Schlemm's canal (iStent), increasing the outflow pathway into the Schlemm's canal; and slow-release antifibrotic drug device (e.g., drug-coated stents). In trabecular bypass surgery a microstent, shunt, or other implants is used to bypass diseased trabecular meshwork or to restore existing outflow pathways. Examples of the trabecular shunts include the EyePass™ glaucoma implant (GMP Companies Inc., FL, USA) and the Glaukos trabecular bypass shunt (iStent). The EyePass glaucoma device is a long, Y-shaped silicone tube with an inner diameter of approximately 1.25 mm and an outer diameter of 2.5 mm, allowing it to fit through the lumen of Schlemm's canal. The trabecular micro-bypass recently developed by Glaukos Corporation (CA, USA) involves gonioscopic surgical treatment to connect the anterior chamber with Schlemm's canal. This avoids conjunctival trauma, in contrast to the other surgical procedures that often traumatize the conjunctiva. The stent is made from titanium and coated with heparin. It is approximately 1 mm in length, and attached to a single-use applicator.

As shown in FIG. 8, anterior ophthalmic surgery involves placement of a small shunt 850 inside the eye in order to alleviate eye pressure, allowing direct communication between the anterior chamber 860 and Schlemm's canal 830, through the trabecular meshwork 820 and juxtacanilicular tissue (JCT) 870, which together provide a barrier for the outflow of aqueous humor. The anterior chamber is traversed with the combination tool, which includes an extendible gripper applicator that holds the implant. The trabecular meshwork is located with OCT real-time imaging and the grippers are extended from the tool, driving the leading edge of the device through the trabecular meshwork into Schlemm's canal at the nasal position (3-4 o'clock angle for the right eye and 9-8 o'clock angle for the left eye). The tip of the implant is directed inferiorly. Traditionally, confirmation of stent entry into Schlemm's canal has been done postoperatively with ultrabio-microscopy or with the surgeon's opinion at the time of surgery, but the combination tool 880 can be used to provide real-time feedback within the forward OCT field of view 885.

Another indication of the proper placement of the stent is blood reflux from Schlemm's canal through the stent. The grippers that extend from the tool 880 can be manipulated within the OCT field 880 as described above, and the device is released by pushing the button 890 on the combination tool 880; the button 890 is mechanically or electrically coupled to the grippers to cause release of the device when pressed. The grippers may then be withdrawn (automatically or manually) into the body of the tool 880. The anterior chamber is flushed of any refluxed blood, and a high-magnification examination along with OCT examination is performed to confirm that the base of the implant is parallel with the circumferential axis of Schlemm's canal. Visualizing the trabecular bypass surgery as well as the surrounding anatomy with the combination tool 880 during surgery can help avoid surgical risks such as poor placement (the stent can fall out if the barbs are not oriented properly) or choroidal hemorrhage (which can happen by surgical error such as mispositioning of the implant).

Cataract surgery is commonly performed on patients with glaucoma or early-stage glaucoma. This procedure allows the surgeon to use the larger holes already required for this surgery (as opposed to trab surgery alone, which tends to involve smaller holes in the cornea), an advantage since larger tools can be inserted in larger holes. New stents have been developed for placement before or at the end of a cataract case. Following uncomplicated cataract extraction, acetylcholine is injected into the anterior chamber to constrict the pupil. A viscoelastic agent is injected to maintain the anterior chamber, while providing more clearance in the insertion angle. As the stent is designed for nasal placement, and cataract surgery is performed on the temporal side, the patient's head must be repositioned. The angle is inspected with a gonioprism to ensure that there is a good view for nasal implantation. Using a combination tool that includes an OCT probe and a surgical introducer allows surgeons to better visualize the anterior chamber in order to optimize device placement, making the treatment of glaucoma faster, safer, and less expensive than currently available modalities while providing a low-cost instrument utilized for cataract surgery.

Embodiments of the present invention facilitate treatment of elevated intraocular pressure in a manner that is safer (since better visualization leads to better placement of implants or treatment of tissue, e.g., using a laser), simpler, more effective, and more disease site-specific. Furthermore, the combination tool can be used in small incisions with diameters smaller than traditional surgical ports (<2 mm) and thus causes minimal impact on the tissues.

Translimbal Device Insertion with Combination Tool

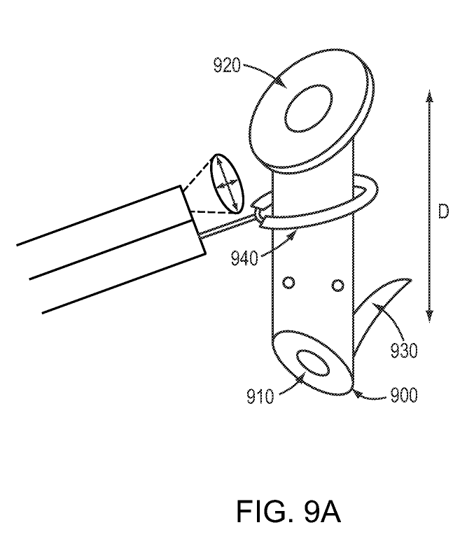

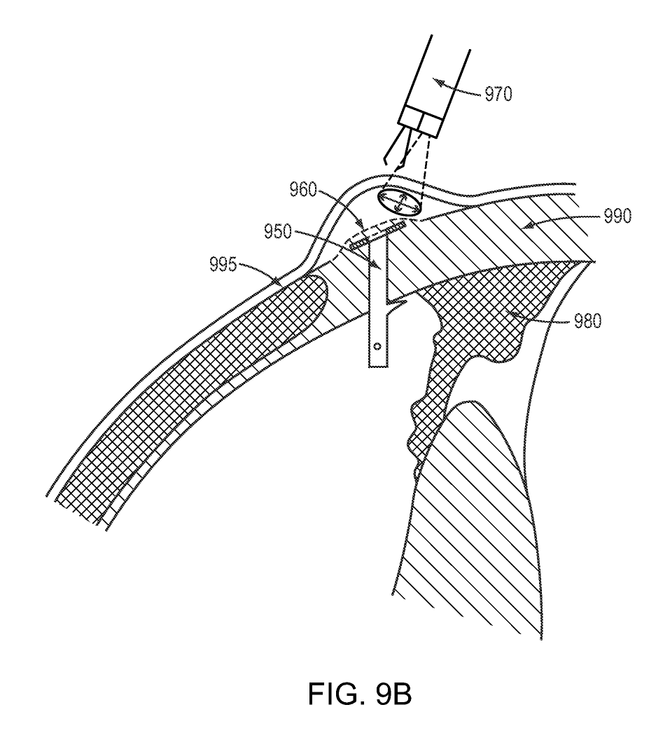

FIG. 9A depicts a translimbal implant 900 used for glaucoma drainage. The implant is approximately 3 mm long and consists of a stainless steel tube with an outer diameter of 400 μm. It has a beveled, sharpened, rounded tip 910 at the proximal end and an angled, flanged plate 920 at the distal end to prevent dislocation into the anterior chamber. An inner spur 930 conforms to the anatomy of the sclera; the distance D between the inner spur 930 and the flanged plate 920 correlates to the scleral thickness at the site of implantation. Surgical complications have been reported when the implant was placed under the conjunctiva, rather than the sclera. An embodiment of this invention provides a customized clasp 940 with a handheld tube for different implants, and thus giving the surgeon a disposable device for easy visualization and implantation in the patient.

With reference to FIG. 9B, the implant 950 is placed under a scleral flap 960. The scleral flap, the roof of Schlemm's canal, was meant to offer resistance to aqueous flow in order to prevent early hypotony and decrease the risk of conjunctival erosion. The combination tool 970 can be used to see through the scleral hole and quickly visualize the location of the iris 980, as well as other anatomical landmarks that are not necessarily apparent on normal light microscopy such as sclera 990 and conjunctiva 995.

### Suprachoroidal Drainage Devices

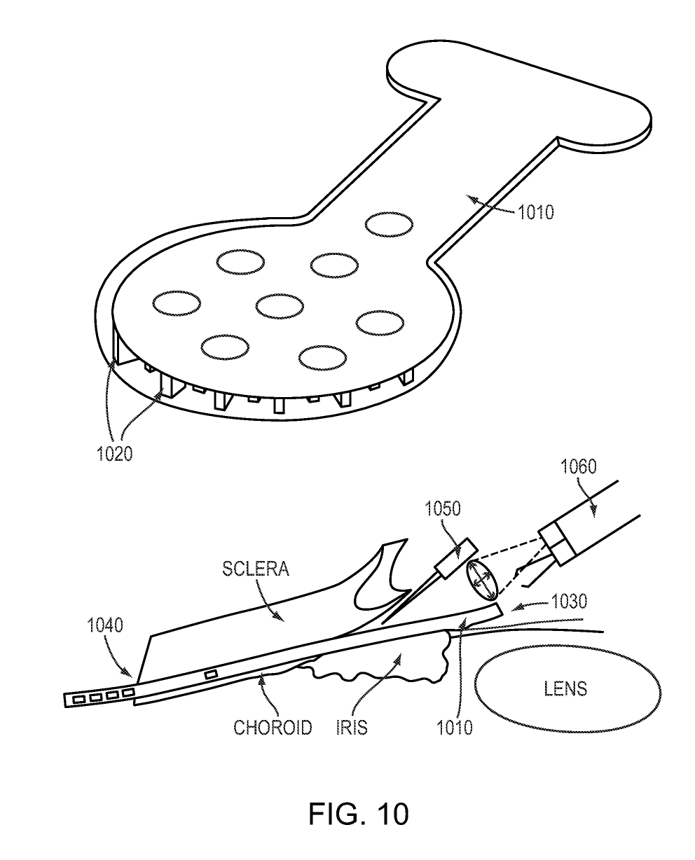

A suprachoridal drainage device, e.g., one or more gold microshunts or Aquashunts, is used to drain the aqueous humor from the anterior chamber into the suprachoroidal space, a potential space between the choroid and sclera. Gold microshunts, developed by DeepLight® Glaucoma Treatment System (SOLX, Inc., Waltham, Mass.), are designed to reduce intraocular pressures (IOPs) without a bleb. FIG. 10 depicts a gold microshunt 1010 containing many microtubules 1020 that form a channel and bridge the anterior chamber 1030 to the suprachoroidal space 1040, ultimately controlling the outflow of the aqueous humor. A typical gold microshunt is a flat 24-karat gold implant that is 3 mm wide, 6 mm long and approximately as thick as human hair. The aqueous fluid from the anterior chamber is directed through the tiny channels and exits the shunt directly into the suprachoroidal space. The pressure gradient that naturally exists between the anterior chamber and suprachoroidal space creates a constant flow of aqueous fluid through the gold shunt.

Implantation of the gold shunt involves a 4-mm, formix-based conjunctival incision. A 3.5-mm scleral cutdown (vertical scleral incision) is created 2 mm posterior from the limbus. A dissection is carried out to the depth where the choroid is visible through the thin layer of sclera. A scleral pocket at 95% depth is created, which is directed anteriorly toward the scleral spur. The vertical cutdown incision is deepened into the choroidal space and viscoelastic material can be administered. An incision is made into the anterior chamber at the level of the scleral spur through the previously created pocket. The gold shunt is then inserted through the scleral incision into the anterior chamber using a “push-then-pull” technique. In order to position the shunt into the suprachoroidal space, the two posterior lateral tabs of the device are tucked into the suprachoroidal space using a sharp 27-gauge needle 1050. This positioning can be visualized with a combination tool 1060. The gold shunt strongly reflects the light signal at the wavelength generated by the OCT and thus is easily visualized on the OCT display. Traditionally, intraoperative gonioscopy has been used to confirm the proper positioning of the shunt in the anterior chamber, but this now can be performed using the combination tool, from the opposite side of the implant with the tool entering through a clear corneal incision; for example, if the implant is placed on the temporal side, the combination tool can be inserted through the clear cornea on the nasal side. The combination tool can be used with opaque dyes (although this is not required) to visualize the anterior drainage openings (which should be visible) and look for the presence of any posterior drainage holes (which should not be seen).

### Aquashunt

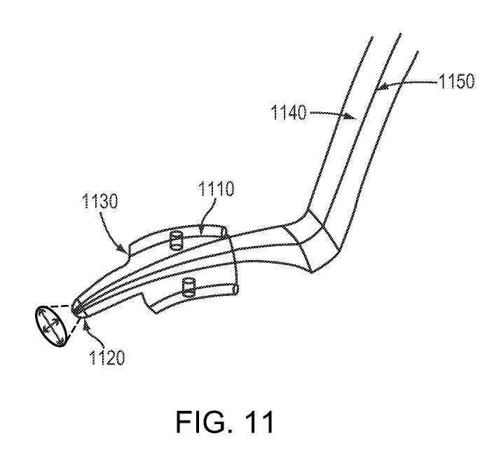

The Aquashunt, developed by OPKO Instrumentation, LLC, has demonstrated a reduction in IOP and its biocompatibility in rabbits. The Aquashunt lowers IOP by shunting aqueous humor from the anterior chamber to the suprachoroidal space. The device is made of a thermoplastic polymer and has a single central channel that conducts aqueous humor into the suprachoroidal space. With reference to FIG. 11, the insertion head of the shunt 1110 has a shearing leading edge 1120 that facilitates entry of the device into the anterior chamber. The shoulders 1130 of the device act as a positive stop and seal the entrance to the anterior chamber. An insertion tool 1140, which also acts as an obturator, is used to insert the device. The obturator prevents clogging of the lumen of the shunt during the advancement of the Aquashunt into the anterior chamber. An A-scan or a B-scan OCT probe 1150 is integrated into the insertion device and provides better visualization and real-time feedback during the surgery. An A-scan OCT probe can be formed by a single fiber with a collimating tip (e.g., via GRIN fiber tip or GRIN lens), while a probe with B-scan capabilities may be formed by laterally moving the tip of an A-scan fiber back and forth using mechanical elements and/or arrangements used in endoscoping imaging (for example, piezo elements, proximate to the fiber tip, that vibrate when a voltage is applied), electromagnetic actuation, rotating-angle cut lenses, or a simple mechanical linkage (e.g., a plastic rod or stiff wire) integrated into the fiber that is pushed, pulled, twisted, rotated, or otherwise moved by a source external to the inserter (e.g., by a motor or pneumatic drive in the insertion tool's handle).

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. A probe assembly comprising an elongated member insertable through an incision in a patient's body, the probe assembly comprising:
an optical coherence tomography (OCT) probe having a field of view; and
adjacent to the probe, an instrument for performing manipulations at least within the OCT field of view.

2. The assembly of claim 1 wherein the OCT probe is a forward-imaging probe.

3. The assembly of claim 1 wherein the OCT probe is a side-imaging probe.

4. The assembly of claim 1 wherein the OCT probe is an angled-imaging probe.

5. The assembly of claim 1 wherein the instrument is configured for movement from a body of the probe assembly into the field of view.

6. The assembly of claim 1 wherein the OCT probe and the instrument are contained within the body.

7. The assembly of claim 1 wherein the manipulations comprise at least one of cutting, gripping, cauterizing or ablating.

8. The assembly of claim 7 wherein the instrument is configured for arcuate extension from the probe assembly into the field of view.

9. The assembly of claim 8 wherein the instrument is a set of retractable grippers.

10. The assembly of claim 1 wherein the instrument is configured to manipulate tissue within the field of view.

11. The assembly of claim 1 wherein the instrument is configured to manipulate an implant within the field of view.

12. The assembly of claim 11 wherein the instrument is a set of forceps.

13. The assembly of claim 11 wherein the instrument comprises an extendible and directional moving element for facilitating rotation and orientation of the implant.

14. The assembly of claim 1 wherein the instrument is configured to deploy an implant within the field of view.

15. The assembly of claim 1 wherein the instrument is an output of a laser.

16. The assembly of claim 1 wherein the instrument is an applicator for an implantable item.

17. The assembly of claim 1 wherein the instrument is a retractable blade.

18. The assembly of claim 17, wherein the retractable blade is made of at least one of metal, silicon, ceramic, or plastic.

19. The assembly of claim 1 wherein the instrument is a cutter.

20. The assembly of claim 1 wherein the instrument is an RF ablation probe.

21. The assembly of claim 1 wherein the instrument is a cautery tool.

22. A system comprising:
a. a probe assembly comprising an elongated member insertable through an incision in a patient's body, the probe assembly comprising:
(i) an optical coherence tomography (OCT) probe having a field of view; and
(ii) adjacent to the probe, an instrument for performing manipulations at least within the OCT field of view;
b. an OCT imaging engine optically coupled to the OCT probe; and
c. a user-controlled actuator remote from the instrument and operatively coupled thereto, the actuator controlling operation of the instrument.

23. The system of claim 22 wherein the OCT probe is a forward-imaging probe.

24. The system of claim 22 wherein the OCT probe is a side-imaging probe.

25. The system of claim 22 wherein the OCT probe is an angled-imaging probe.

26. The system of claim 22 wherein the actuator communicates pneumatically with the instrument along a fluid path thereto.

27. The system of claim 22 wherein the actuator communicates mechanically with the instrument.

28. The system of claim 27 wherein the actuator is located on a user handle, and further comprising a wire connecting the actuator to the instrument and facilitating extension and retraction thereof.

29. A method for performing manipulations during surgery, the method comprising:
inserting a probe assembly adjacent to or into a target;
capturing an OCT image of the target via the probe, the OCT image having a field of view; and
performing manipulations on at least one of tissue or an implantable device within the field of view.

30. The method of claim 29, wherein the OCT image is obtained via image-gathering optics and the manipulations are performed by an instrument, the image-gathering optics and the instrument being contained within the probe assembly.

31. The method of claim 29, wherein the manipulations comprise at least one of cutting, gripping, cauterizing, or ablating the target.
